(19) United States
(12) Patent Application Publication
(10) Pub. No.: US 2021/0161808 A1
(43) Pub. Date: Jun. 3, 2021

(54) Nasal Drug Delivery System of Brivaracetam or Salt Thereof

(71) Applicant: Zenvision Pharma LLP, Mumbai (IN)

(72) Inventors: Sivakumar Venkata Bobba, Navi Mumbai (IN); Bhimrao Jadhav, Navi Mumbai (IN); Dhananjay Shinde, Navi Mumbai (IN); Sunil Pophale, Navi Mumbai (IN)

(21) Appl. No.: 17/261,936

(22) PCT Filed: Jul. 11, 2019
(86) PCT No.: PCT/IB2019/055917

§ 371 Date: Jan. 21, 2021

(30) Foreign Application Priority Data
Jul. 24, 2018 (IN) 201821027730

(51) Int. Cl. A61K 9/00; A61K 31/4015; A61K 47/10; A61K 47/26; A61K 9/08

(52) U.S. Cl. CPC A61K 9/0043; A61K 31/4015; A61K 9/08; A61K 47/26; A61K 47/10

(57) ABSTRACT

The present disclosure relates to the intranasal drug delivery system or composition of Brivaracetam or salt thereof. The nasal drug delivery Brivaracetam or salt thereof preferably in the form of Nasal sprays provide more bioavailability better onset of action, lesser adverse effects; better ease of self-administration and better patient compliance in the treatment of partial-onset seizures.

### FIELD OF THE INVENTION

The present invention relates to nasal drug delivery system of Brivaracetam or salt thereof for the treatment of partial-onset seizures.

### BACKGROUND OF THE INVENTION

Epilepsy is a group of neurological disorders characterized by epileptic seizures. Epileptic seizures are episodes that can vary from brief and nearly undetectable periods to long periods of vigorous shaking. These episodes can result in physical injuries, including occasionally broken bones.

The cause of most cases of epilepsy is unknown. Some cases occur as the result of brain injury, stroke, brain tumors, infections of the brain, and birth defects through a process known as epileptogenesis. Epileptic seizures are the result of excessive and abnormal neuronal activity in the cortex of the brain

Epilepsy is usually treated with daily medication once a second seizure has occurred, while medication may be started after the first seizure in those at high risk for subsequent seizures. There are a number of medications available including Brivaracetam, Acetazolamide, Carbamazepine, Clobazam, Clonazepam, Eslicarbazepine acetate, Ethosuximide, Everolimus, Gabapentin, Lacosamide, Lamotrigine, Levetiracetam, Oxcarbazepine, Perampanel, Phenobarbital, Phenytoin, Piracetam, Pregabalin, Primidone, Rufinamide, Sodium valproate, Stiripentol, Tiagabine, Topiramate, Valproic acid and Vigabatrin.

Brivaracetam is a third-generation antiepileptic racetam derivative and a 4-n-propyl analogue of levetiracetam. Chemically brivaracetam is (2S)-2-[(4R)-2-oxo-4-propyltetrahydro-1H-pyrrol-1-yl] butanamide and its molecular weight is 212.29. Its empirical formula is C₁₁H₂₀N₂O₂. Brivaracetam is represented by compound of structural formula I

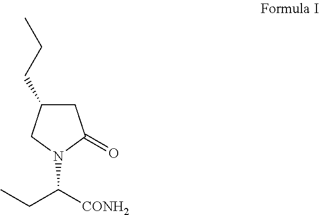

Brivaracetam is a white to off-white crystalline powder. It is very soluble in water, buffer (pH 1.2, 4.5, and 7.4), ethanol, methanol, and glacial acetic acid. It is freely soluble in acetonitrile and acetone and soluble in toluene. It is very slightly soluble in n-hexane.

Brivaracetam tablets of UCB INC has been approved in USA as on May 12, 2016 under the trade name BRIVIACT® and is available in the strength of 10 mg, 25 mg, 50 mg, 75 mg, 100 mg. The product is indicated for the treatment of partial-onset seizures in patients 4 years of age and older.

Brivaracetam oral solution of UCB INC has been approved in USA as on May 12, 2016 under the trade name BRIVIACT® and is available in the strength of 10 mg/ml. The product is indicated for the treatment of partial-onset seizures in patients 4 years of age and older.

Brivaracetam solution intravenous of UCB INC has been approved in USA as on May 12, 2016 under the trade name BRIVIACT® and is available in the strength of 50 mg/5 ml (10 mg/ml). The product is indicated for the treatment of partial-onset seizures only in adult patients (16 years of age and older).

U.S. Pat. No. 6,784,197 discloses compound Brivaracetam or salt thereof. The said patent also discloses generically compositions of brivaracetam or salt thereof can be administered oral or parenteral route i.e., intravenously, intramuscularly or subcutaneously, intrathecally. The said patent further generically discloses pharmaceutical compositions suitable for oral administration can be solids or liquids and can be in the form of tablets, pills, dragees, gelatin capsules, solutions, syrups, and the like. However, U.S. Pat. No. 6,784,197 does not disclose or teaches generically or specifically nasal drug delivery system for brivaracetam or salt thereof.

U.S. Pat. No. 8,492,416 discloses a method for treatment of epilepsy, seizure disorders and convulsions comprising administering to a mammal in need thereof a therapeutically effective amount of Brivaracetam. However, U.S. Pat. No. 8,492,416 does not disclose or teaches generically or specifically nasal drug delivery system for Brivaracetam or salt thereof.

The product known in the prior art for Brivaracetam or salt thereof is available in the form of oral tablet, solution and intravenous solution. Due to their high dosage, commercially available product and product known in the prior art for Brivaracetam or salt thereof suffers from adverse reactions like suicidal behavior and ideation, neurological adverse reactions like somnolence, fatigue, dizziness, and disturbance in coordination, psychiatric adverse reactions include irritability, anxiety, nervousness, aggression, belligerence, anger, agitation, restlessness, depression, depressed mood, tearfulness, apathy, altered mood, mood swings, affect liability, psychomotor hyperactivity, abnormal behavior, adjustment disorder, hallucination, paranoia, acute psychosis, and psychotic behavior, hypersensitivity reactions bronchospasm and angioedema.

Also, product known in the prior art for oral tablet, solution shows slowed absorption with high fat meal; therefore delayed onset of action, also susceptible to first pass metabolism and gastric degradation. Further product known in the prior art for intravenous solution does not provide patient compliance since its administration is painful and dependent on other healthcare professional.

Thus there is an unmet need in the art to provide drug delivery system which will provide better onset of action, by pass first pass metabolism and gastric degradation, minimum dosages with lesser adverse effects, more bioavailable, with better ease of self-administration and which provides better patient compliance in the treatment of partial-onset seizures.

Accordingly applicant of the present invention provides a nasal drug delivery system of Brivaracetam or salt thereof which provides better onset of action, by pass first pass metabolism and gastric degradation, minimum dosages with lesser adverse effects, offer larger nasal mucosal surface area for drug absorption, more bioavailable, with better ease of self-administration and better patient compliance in the treatment of partial-onset seizures.

### OBJECTS OF THE INVENTION

Accordingly it is an object of the present invention to provide an intranasal drug delivery system or composition of Brivaracetam or salt thereof.

It is another object of the present invention to provide an intranasal drug delivery system or composition of Brivaracetam or salt thereof which provides better onset of action

It is another object of the present invention to provide an intranasal drug delivery system or composition of Brivaracetam or salt thereof which is more bioavailable.

It is another object of the present invention to provide an intranasal drug delivery system or composition of Brivaracetam or salt thereof with low dose and which provides less adverse effects in the treatment of partial-onset seizures.

It is another object of the present invention to provide an intranasal drug delivery system or composition of Brivaracetam or salt thereof which provides better ease of self-administration and patient compliance in the treatment of partial-onset seizures.

### SUMMARY OF THE INVENTION

A first aspect of the present invention is to provide an intranasal drug delivery system or composition of Brivaracetam or salt thereof.

In another aspect of the present invention is to provide an intranasal drug delivery system or composition of Brivaracetam or salt thereof in the form of Nasal sprays, Nasal drops, Nasal gels and Nasal powders.

In another aspect of the present invention is to provide an intranasal drug delivery system or composition of Brivaracetam or salt thereof preferably in the form of Nasal sprays.

In another aspect of the present invention is to provide an intranasal drug delivery system or composition of Brivaracetam or salt thereof in the form of Nasal sprays, Nasal drops, Nasal gels and Nasal powders along with one or more pharmaceutically acceptable excipient.

In another aspect of the present invention is to provide process of manufacturing an intranasal drug delivery system or composition of Brivaracetam or salt thereof in the form of Nasal sprays, Nasal drops, Nasal gels and Nasal powders along with one or more pharmaceutically acceptable excipient.

In another aspect of the present invention is to provide method of treating partial-onset seizures by administration of an intranasal drug delivery system or composition of Brivaracetam or salt thereof in the form of Nasal sprays, Nasal drops, Nasal gels and Nasal powders.

### DETAIL DESCRIPTION OF THE INVENTION

The present invention relates to an intranasal drug delivery system or composition of Brivaracetam or salt thereof.

Intranasal drug delivery system according to present invention is an intranasal pharmaceutical composition for administration of drug through nasal route. Nasal administration includes administering the compositions into nostrils of the nose to the mucous membranes of the nasal passage or nasal cavity of the mammal.

The nasal mucosa is a potential route of drug administration for achieving faster and higher levels of absorption. This is mainly due to the large surface area of the nasal mucosa, its porous endothelial membrane, the high amount of blood flow in the mucosa and its ability to bypass the first-pass hepatic metabolism.

The pharmaceutical composition of Brivaracetam or salt thereof according to the present invention may be in the form of Nasal sprays, Nasal drops, Nasal gels and Nasal powders.

The pharmaceutical composition of Brivaracetam or salt thereof may be preferably in the form of Nasal sprays.

The pharmaceutical composition of Brivaracetam or salt thereof according to present invention in the form of Nasal sprays, Nasal drops, Nasal gels and Nasal powders may comprise one or more pharmaceutically acceptable excipients.

The pharmaceutical composition of the present invention can comprise any suitable amount of the Brivaracetam or salts thereof. The weight percentage of Brivaracetam ranges from 0.01% to 20%, preferably 0.05% to 10% based on the total volume of composition.

The one or more pharmaceutically acceptable excipients according to present invention may be selected from the group consisting of penetration enhancer, buffering agents, antioxidants, preservatives, surfactants, bioadhesive polymers or gelling agent, solvents, solubilizers, pH adjustifier, tonicity adjustifier, humectants and enzyme inhibitors.

The examples of penetration enhancer include but not limited to polyethylene glycol, Polysorbate, pyrrolidones, alkyl methyl sulphoxides, Dodecyl azacycloheptan-2-one, Cyclodextrins, Fusidic acid derivatives, Phosphatidylcholines, Trihydroxy salts (glycol- and taurocholate), Salicylates, Ethylenediaminetetraacetic acid (EDTA), Oleic acid, Caprylate (C8), Caprate (C10), Laurate, Lysophosphatidylcholine, Didecanoyl, Carbenozolone, Glycyrrhizinate, α, β, and γ-cyclodextrins and their derivatives, n-glycofurols and n-ethylene glycols, Polyoxyethylene-9-lauryl ether (Laureth-9), Saponin or combinations thereof. The amount of penetration enhancers present in the composition ranges from about 0.5-50%; preferably about 5-40% based on the total volume of the composition.

The examples of buffering agent include but not limited phosphate, citrate, tris, succinate, histidine, glycine, arginine, malic, tartaric, acetic, benzoic, gluconic, glyceric, lactic, aconitic, adipic, ascorbic, carbonic, glutamic, ammonium chloride, Sodium acetate trihydrate, sodium chloride, triethanolamine or combinations thereof. The amount of buffering agent present in the composition ranges from about 0.01-3% preferably about 0.05-2.5% based on the total volume of the composition.

The examples of antioxidants include but not limited to sodium metabisulfite, sodium bisulfate, butylated hydroxytoluene and tocopherol or combinations thereof. The amount of antioxidant present in the composition ranges from about 0.0001-10%; preferably about 0.001-5% based on the total volume of the composition.

The examples of preservatives include but not limited to parabens, benzalkonium chloride, phenyl ethyl alcohol, EDTA and benzoyl alcohol or combinations thereof. The amount of preservative present in the composition ranges from about 0.01-5%; preferably about 0.05-2.5% based on the total volume of the composition.

The examples of surfactant include but not limited to sodium glycocholate, sodium taurocholate, polyoxyethylene lauryl ether, polyacrylic acid gel, Sodium lauryl sulfate, Polysorbate and sodium deoxycholate or combinations thereof.

The examples of bioadhesive polymers or gelling agent include but not limited to guar gum, starch, hydroxyl ethyl cellulose, sodium hyaluronate, sodium alginate, polycarbophil, methyl cellulose, dextran, hydroxypropylmethyl cellulose, hydroxypropyl cellulose, sodium carboxy methyl cellulose, carbopol and chitosan or combinations thereof.

The examples of solvents include but not limited to water, ethanol, polyethylene, propylene glycols or combinations thereof.

The examples of solubilizers include but not limited to glycols, small quantities of alcohol, Transcutol (diethylene glycol monoethyl ether), medium chain glycerides and Labrasol (saturated polyglycolyzed C8-C10 glyceride) or combinations thereof.

The examples of pH adjustifier according to present invention include but not limited to hydrochloric acid, citric acid, sodium hydroxide, sodium carbonate, lactic acid or combinations thereof.

The examples of tonicity adjustifier include but not limited to anhydrous dextrose, sodium chloride, glucose, sorbitol, mannitol and xylitol or combinations thereof.

The examples of humectants include but not limited to glycerine, sorbitol and mannitol or combinations thereof.

The examples of enzyme inhibitors include but not limited to bestatin, amastatin, boroleucine, protein and peptides, fusidic acids, and bile salts or combinations thereof.

In another aspect of the present invention is to provide process of manufacturing pharmaceutical composition of Brivaracetam or salt thereof in the form of Nasal sprays, Nasal drops, Nasal gels and Nasal powders. The process of manufacturing pharmaceutical composition of Brivaracetam or salt involves steps of dissolving or suspending Brivaracetam in solvents and mixing the same with one or more pharmaceutically acceptable excipients selected from the group consisting of penetration enhancer, surfactants, bioadhesive polymers or gelling agent, solvents, buffers, solubilizers, pH adjustifier, tonicity adjustifier, preservatives, antioxidants, humectants and enzyme inhibitors.

The concentration of Brivaracetam or salt thereof, excipients and manufacturing process has been optimized in such way that nasal drug delivery system of Brivaracetam or salt thereof according to present invention provide better absorption results in more bioavailability better onset of action, lesser adverse effects and better patient compliance in the treatment of partial-onset seizures

The nasal drug delivery system of Brivaracetam or salt thereof according to present invention by passes first pass metabolism and gastric degradation, offer larger nasal mucosal surface area for drug absorption results in more bioavailability, provides better ease of self-administration and better patient compliance in the treatment of partial-onset seizures.

The nasal drug delivery system of Brivaracetam or salt thereof according to present invention effectively treats partial-onset seizures; wherein Brivaracetam or salt thereof treats partial-onset seizures by high and selective affinity for synaptic vesicle protein 2A (SV2A) in the brain.

The pharmaceutical compositions of the present invention were evaluated for the parameters like appearance, pH, osmolality, viscosity, assay and found to be in the compliance.

The pharmaceutical compositions of Brivaracetam or salt thereof according to present invention can be packaged in suitable plastic or glass containers. Further pharmaceutical composition is delivered in to the nasal passages through nasal spray, Squeeze bottles, Drops delivered with pipette, Delivery of liquid with rhinyle catheter and squirt tube, Metered-dose spray pumps, Single- and duo-dose spray devices, Nasal pressurized metered-dose inhalers

### EXAMPLES

The following Examples are provided solely for illustrative purposes and are not meant to limit the invention in any way.

### Example 1: Brivaracetam Nasal Spray

| Sr. No. | Ingredients | mg/ml | % |
|---|---|---|---|
| 1. | Brivaracetam | 25.00 | 2.50 |
| 2. | Polyethylene glycol | 200.00 | 20.00 |
| 3. | Polysorbate | 100.00 | 10.00 |
| 4. | Sodium acetate trihydrate | 5.00 | 0.50 |
| 5. | Butylated hydroxytoluene | 0.02 | 0.002 |
| 6. | Methyl paraben | 4.00 | 0.40 |
| 7. | Purified water | q.s. | q.s. |

### Manufacturing Process:

1. Methyl paraben and butylated hydroxytoluene were dissolved in part quantity of purified water under stirring and temperature.
2. Cooled solution of step 1 to room temperature and added sodium acetate trihydrate, polyethylene glycol and polysorbate and mixed to form clear solution.
3. Added Brivaracetam to solution of step 2 and mixed to dissolve completely.
4. Volume was made up to required quantity using purified water.
5. Fill in suitable container or packing material.

### Example 2: Brivaracetam Nasal Spray

| Sr. No. | Ingredients | mg/ml | % |
|---|---|---|---|
| 1. | Brivaracetam | 25.00 | 2.50 |
| 2. | Polyethylene glycol | 200.00 | 20.00 |
| 3. | Polysorbate | 100.00 | 10.00 |
| 4. | Sodium chloride | 9.00 | 0.90 |
| 5. | Butylated hydroxytoluene | 0.02 | 0.002 |
| 6. | Methyl paraben | 4.00 | 0.40 |
| 7. | Purified water | q.s. | q.s. |

### Manufacturing Process:

1. Methyl paraben and butylated hydroxytoluene were dissolved in part quantity of purified water under stirring and temperature
2. Cooled solution of step 1 to room temperature and added sodium chloride, polyethylene glycol and polysorbate and mixed to form clear solution.
3. Added Brivaracetam to solution of step 2 and mixed to dissolve completely.
4. Volume was made up to required quantity using purified water.
5. Fill in suitable container or packing material.

### Example 3: Brivaracetam Nasal Spray

| Sr. No. | Ingredients | mg/ml | % |
|---|---|---|---|
| 1. | Brivaracetam | 25.00 | 2.50 |
| 2. | Polyethylene glycol | 100.00 | 10.00 |
| 3. | Polysorbate | 100.00 | 10.00 |
| 4. | Sodium acetate trihydrate | 1.64 | 0.164 |
| 5. | Butylated hydroxytoluene | 0.02 | 0.002 |
| 6. | Methyl paraben | 4.00 | 0.40 |
| 7. | Purified water | q.s. | q.s. |

### Manufacturing Process:

1. Methyl paraben and butylated hydroxytoluene were dissolved in part quantity of purified water under stirring and temperature.
2. Cooled solution of step 1 to room temperature and added sodium acetate trihydrate, polyethylene glycol and polysorbate and mixed to form clear solution.
3. Added Brivaracetam to solution of step 2 and mixed to dissolve completely.
4. Volume was made up to required quantity using purified water.
5. Fill in suitable container or packing material.

### Example 4: Brivaracetam Nasal Spray

| Sr. No. | Ingredients | mg/ml | % |
|---|---|---|---|
| 1. | Brivaracetam | 25.00 | 2.50 |
| 2. | Polyethylene glycol | 200.00 | 20.00 |
| 3. | Polysorbate | 50.00 | 5.00 |
| 4. | Sodium acetate trihydrate | 1.64 | 0.164 |
| 5. | Butylated hydroxytoluene | 0.02 | 0.002 |
| 6. | Methyl paraben | 4.00 | 0.40 |
| 7. | Purified water | q.s. | q.s. |

### Manufacturing Process:

1. Methyl paraben and butylated hydroxytoluene were dissolved in part quantity of purified water under stirring and temperature.
2. Cooled solution of step 1 to room temperature and added sodium acetate trihydrate, polyethylene glycol and polysorbate and mixed to form clear solution.
3. Added Brivaracetam to solution of step 2 and mixed to dissolve completely.
4. Volume was made up to required quantity using purified water.
5. Fill in suitable container or packing material.

### Example 5: Brivaracetam Nasal Spray

| Sr. No. | Ingredients | mg/ml | % |
|---|---|---|---|
| 1. | Brivaracetam | 10.00 | 1.00 |
| 2. | Polyethylene glycol (high | 200.00 | 20.00 |
| | molecular weight) | | |
| 3. | Polysorbate | 50.00 | 5.00 |
| 4. | Sodium acetate trihydrate | 1.64 | 0.164 |
| 5. | Butylated hydroxytoluene | 0.02 | 0.002 |
| 6. | Methyl paraben | 4.00 | 0.40 |
| 7. | Purified water | q.s. | q.s. |

### Manufacturing Process:

1. Methyl paraben and butylated hydroxytoluene were dissolved in part quantity of purified water under stirring and temperature.
2. Cooled solution of step 1 to room temperature and added sodium acetate trihydrate, polyethylene glycol (high molecular weight) and polysorbate and mixed to form clear solution.
3. Added Brivaracetam to solution of step 2 and mixed to dissolve completely.
4. Volume was made up to required quantity using purified water.
5. Fill in suitable container or packing material.

### Example 6: Brivaracetam Nasal Spray

| Sr. No. | Ingredients | mg/ml | % |
|---|---|---|---|
| 1. | Brivaracetam | 25.00 | 2.50 |
| 2. | Polyethylene glycol | 50.00 | 5.00 |
| 3. | Polysorbate | 50.00 | 5.00 |
| 4. | Sodium acetate trihydrate | 1.64 | 0.164 |
| 5. | Butylated hydroxytoluene | 0.02 | 0.002 |
| 6. | Methyl paraben | 4.00 | 0.40 |
| 7. | Purified water | q.s. | q.s. |

### Manufacturing Process:

1. Methyl paraben and butylated hydroxytoluene were dissolved in part quantity of purified water under stirring and temperature.
2. Cooled solution of step 1 to room temperature and added sodium acetate trihydrate, polyethylene glycol and polysorbate and mixed to form clear solution.
3. Added Brivaracetam to solution of step 2 and mixed to dissolve completely.
4. Volume was made up to required quantity using purified water.
5. Fill in suitable container or packing material.

1. An intranasal pharmaceutical composition comprising Brivaracetam or salt thereof and pharmaceutically acceptable excipients.

2. The pharmaceutical composition according to claim 1, in the form of nasal sprays, nasal drops, nasal gels and nasal powders.

3. The pharmaceutical composition according to claim 2, in the form of nasal sprays.

4. The pharmaceutical composition according to claim 1, wherein the amount of Brivaracetam or salt thereof is in the range of 0.05% to 10% based on the total volume of composition.

5. The pharmaceutical composition according to claim 1, further comprising one or more pharmaceutically acceptable excipients selected from the group consisting of penetration enhancer, buffering agents, antioxidants, preservatives, surfactants, bioadhesive polymers or gelling agent, solvents, solubilizers, pH adjustifier, tonicity adjustifier, humectants and enzyme inhibitors.

6. The pharmaceutical composition according to claim 5, wherein the penetration enhancer is selected from the polyethylene glycol, Polysorbate, pyrrolidones, alkyl methyl sulphoxides, Dodecyl azacycloheptan-2-one, Cyclodextrins, Fusidic acid derivatives, Phosphatidylcholines, Trihydroxy salts (glycol- and taurocholate), Salicylates, Ethylenediaminetetraacetic acid (EDTA), Oleic acid, Caprylate (C8), Caprate (C10), Laurate, Lysophosphatidylcholine, Didecanoyl, Carbenozolone, Glycyrrhizinate, α, ß, and γ-cyclodextrins and their derivatives, n-glycofurols and n-ethylene glycols, Polyoxyethylene-9-lauryl ether (Laureth-9), Saponin, or combinations thereof.

7. The pharmaceutical composition according to claim 6, wherein the penetration enhancer is polyethylene glycol, Polysorbate or combinations thereof.

8. The pharmaceutical composition according to claim 6, wherein amount of penetration enhancer is in the range of 5% to 40% based on the total volume of composition.

9. An intranasal pharmaceutical composition comprising Brivaracetam or salt thereof according to claim 1, for use in the treatment of partial-onset seizures.
